(19) United States
(12) Patent Application Publication
(10) Pub. No.: US 2009/0123998 A1
(43) Pub. Date: May 14, 2009

(54) Signature encoding sequence for genetic preservation

(76) Inventor: Alexey Gennadievich Zdanovsky, Madison, WI (US)

Correspondence Address: Alexey Zdanovsky, 1202 Ann Street, Madison, WI, 53713, US

(21) Appl. No.: 11/428,774

(22) Filed: Jul. 5, 2006

Related U.S. Application Data
(60) Provisional application No. 60/696,366, filed on Jul. 5, 2005.

(51) Int. Cl. C12N 15/00; G10H 1/00

(52) U.S. Cl. 435/320.1; 84/600

(57) ABSTRACT

The present invention describes a method for fabrication of nucleic acid-based medium for storage of socially valuable information. This medium offers the possibility of simple and efficient reproduction of the information and cm be used for preserving information over periods of time, far surpassing those currently achievable by information storage devices utilizing conventional media.

### CROSS-REFERENCE TO RELATED APPLICATIONS

I claim the benefit of provisional application 60/696,366 filed on Jul. 5, 2005 entitled “Signature encoding sequence for genetic preservation”.

### BACKGROUND OF THE INVENTION

There is a great diversity of methods for encoding, encrypting, and identifying information by alphanumeric designation, language, numbering systems, and indicators or identifiers. One of the most common identifiers is bar coding, virtually ubiquitous systems of lines, dots, and other features which may be scanned and interfaced by information processors to retrieve information about inventory, price, location, and other useful parameters. There are hundreds of patents which disclose and claim variations in bar coding technology, U.S. Pat. No. 6,779,665 provides a good summary of bar coding techniques, and discloses a bar coding system as an identifier for molecular interactions on a matrix.

Several patents utilize a readable signature system for identifying and authenticating articles of commerce. For example, U.S. Pat. No. 6,839,453 discloses an image storage vehicle for recording and authenticating autographs, by making the stored information available to the owner of the article so marked, hi U.S. Pat. No. 6,638,593, a more elaborate marking system using dyes invisible in the visual spectrum, but detectable by UV or IR, is employed. A code is written in IR ink which contains an up-converting phosphor which produces a sparkle effect of a certain wavelength. For complete authentication, a garment to which the mark is attached must pass three distinct levels of protection.

U.S. Pat. No. 6,779,665 discloses a genealogy storage kit comprising a plurality of biological sample devices, sample collection devices, and identification devices for preserving multiple biological parameters of individuals for future identity comparisons. The patent describes containers (including scalable bags as disclosed in U.S. Pat. No. 5,101,970) for human blood, saliva, semen, and tissue, and also photographs and a fingerprint recordation device.

The use of DNA to encode messages in language is disclosed in U.S. Pat. No. 6,312,911. An alphanumeric coding system utilizes triplet codons to stand for individual letters and numbers. Triplets are assigned randomly, thus making it exceedingly difficult for a would be reader to decipher the target sequence into a message, if the reader is ignorant of the encryption key. The principal objective of the invention, in fact, is to hide an encrypted message in steganographic fashion within a mass of unrelated DNA.

U.S. Patent Application No, 20030219756 similarly employs a randomized coding key to create an alphanumeric alphabet. The principal objective in this invention is storage of messages in the DNA of living organisms. Finally. P.S. Patent Application No. 20050053968 discloses a information storage system for complex images utilizing software and a set of schemes to encrypt information including a unique 4-base per character key comprised of 256 characters.

### GLOSSARY OF DEFINITIONS

As used herein, the following terms and phrases shall have the meanings assigned below.

“Nucleic acid sequence” means the order of bases contained in covalently linked deoxyribonucleotides or ribonucleotides proceeding 5′ to 3′, and is intended to include and be the equivalent of a corresponding complementary sequence proceeding 3′ to 5′. The term applies to both a notation of the order of bases and to the corresponding physical molecule having that order of bases.

“Social information” means any information relating to human society, the interaction of the individual and the group, or the welfare of human beings as members of society, and distinct from the information about biologic function.

“Elements of meaning” is a unit of thought expressible to and understood by one or more individuals. The term applies to a word as an element of speech, a note on a scale in music, and notations which confer an element of information.

“DNA or RNA strand” means a polynucleotide or polynucleotides made up of a plurality of covalently attached deoxyribonucleotides or ribonucelosides. The terms include both sense and antisense sequences and the duplex formed by complementary annealing.

“Replicon” means an array of genetic elements capable of self-replication with or without the helper functions of a host. The terms includes plasmids and other vectors, viruses, bacteria, and higher order cells such those of plants and animals. If a nucleic acid sequence is integrated into a chromosome of a host organism, then the host is a replicon.

“Flanking sequence” is a nucleic acid sequence located 5′ or 3′ of a target sequence of interest.

“Encrypt” or “encrypted” means to convert (as a body of information) from one system of communication into another.

“Signature” refers to something that serves to set apart or identify.

“Splicable” means a nucleic acid sequence having a site or sites which provide a substrate for an enzyme such as an endonuclease which can cleave the strand at or near the site or sites.

“Cloning cassette” means a nucleic acid sequence having one or more different spliceable sites

“Essential reading frame” means a sequence of codons (such as nucleotide triplets) that is potentially translatable into a polypeptide or the information carrying message.

### SUMMARY OF THE INVENTION

For over 50 years since Watson and Crick first discovered the structure of DNA and the genetic code, study of the structure mid function of nucleic acids has focused on their role in gene expression and control of metabolic processes. The language of DNA consists of the arrangement of four bases contained in nucleotide sequences. In the case of structural genes, bases are arranged in triplets called codons, each codon encoding an amino acid making up the resultant protein. Other sequences are regulatory, and control gene expression. The genome of higher organisms also contains large amount of DNA of no known function. Attention has been focused entirely on the genetic aspects of nucleic acids and their role in life processes.

It is known, however, that most life forms are capable of propagating extra nucleic acids not needed for their normal functions. The process of isolating and inserting such “foreign” DNA into an organism's genome is popularly called “genetic engineering” or cloning. Most of these cloning experiments are designed to alter gene expression in the host to express a gene not normally present. In any case, the goal is fundamentally genetic and the new information inserted is intended as information related to biological function.

In the present invention, nucleic acid sequences are inserted without regard to the genetic content of the sequences, and, any actual genetic effect is purely unintentional. The object of the present invention is to preserve social information. The same assembly of nucleic acid sequences which are arranged to impart the genetic code can also be made to encode social information such as language or music, and thereby impart elements of meaning which constitute messages understandable by the reader, who sequences the DNA, or a protein derived therefrom.

The invention provides a nucleic acid sequence as a media for preserving social information utilizing combinations of the four bases contained in DNA or RNA arranged to form elements of meaning. The method of the invention comprises assigning combinations of these bases (including their chemical analogs) to form elements of meaning, and then synthesizing the sequences into DNA or RNA strands. Preferably the synthesized strand contains 3′ and 5′ flanking sequences spliceable into a functional DNA in a host, and ultimately into a replicon.

Polynucleotides which may encode units of human language containing the four bases of DNA or RNA in which unit combinations of triplets up to nanoplets of base-containing deoxyribonucleotides or ribonucleotides are selected to correspond to the characters of a human language. These are linearly arrayed to form elements of human speech.

In a preferred embodiment, a personalized signature encoded message in a nucleic acid sequence utilizes a continuous sequence of nucleotide (RNA or DNA) triplets, wherein each triplet corresponds (optionally in an open reading frame) to at least one and not more than two degenerate axiom for an amino acid having a conventionally and essentially universally designed single English character, namely a letter symbol, and all other character/letters, and characters of punctuation are assigned randomly from the remaining amino acid degenerate codons, and non-amino acid cottons, in an internally consistent and statistically predicable usage. Preferably, such personalized encoded message nucleic acid sequence has spliceable flanking sequences 5′ and 3′ of the signature to enable integration into a vector, plasmid, or host capable of genetically maintaining it for propagation and preservation.

It is a further object of the invention to incorporate a fragment of any species sought to be genetically preserved in the same molecule together with a signature or identifier sequence, and a specific tag sequence, as hereafter defined This embodiment comprises a signature encoding a nucleic acid sequence, a fragment or fragments to be expressed or quiescently represented in a library of fragments.

In a specific embodiment of the present invention, a signature encoding nucleic acid language base of characters contains a sequence constructed by selecting codons having single English letter symbols assigned to encoded amino acids including codons for phenylalanine, symbolically designated “f”, leucine, symbolically designated “l”, a codon for isoleucine, symbolically designated “i”, the codon for methionine, symbolically designated “m”, a codon for valine, symbolically designated “v”, a codon for serine, symbolically designated “s”, a codon for proline, symbolically designated “p”, a codon for threonine, symbolically designated “t”, a codon for alanine, symbolically designated “a”, a codon for tyrosine, symbolically designated “y”, a codon for histidine, symbolically designated “b”, a codon for glutamine, symbolically designated “q”, a codon for asparagines, symbolically designated “n”, a codon for glutamic acid, symbolically designated “e”, a codon for cysteine, symbolically designated “c”, a codon for tryptophane, symbolically designated “w”, a codon for arginine, symbolically designated “r”, a codon for glycine, symbolically designated “g”, and a codon selected arbitrarily, but consistently and predictably from all remaining codons and assigned to a character consisting of “b”, “j”, “o”, “x”, and “z”, or to an element of punctuation, “space”, “!”, “.”, “,”, “upper case”, “?”, and “-”.

The codons selected consist of one and not more than two of the available codons for each amino acid designated letter or a codon selected randomly for a letter not part of the conventional letter symbol lexicon. So, a codon for phenylalanine is selected from one and not more than two of TTT and TTC, for leucine from TTA. TTG, CTT, CTC, CTA, and CTG, for isoleucine from ATT, ATC, and ATA, for methionine uniquely (torn ATG, for valine from GTT, GTC, GTA, and GTG, for serine from TCT, TCC, TCA, AGT, and AGC, for proline from CCT, CCC, CCA, and CCG, for threonine from ACT, ACC, ACA, and ACG, for alanine from GCT, GCC, CCA, and GCG, for tyrosine from TAT and TAC, for histidine from CAT and CAC, for glutamine from CAA and CAG, for asparagine from AAT and AAC, for glutamic acid from GAA and GAG, for cysteine from TGT and TGG, for tryptophane uniquely from TGC, for arginine from CGT, CCC, GGA, CGG, AG A, and AGO, and for glycine from GGT, GGC, GGA, and GGG, respectively.

In a second example of a signature nucleic acid sequence encoding social information, the conventionally designated amino acid codons can be arranged to correspond to the notes of a music scale. The sequence comprises selecting codons having conventionally designated single letter symbols for amino acids including a codon for cystine symbolically designated “c” consisting of one of TGT or TGC, a codon for cystine symbolically designated “c*” consisting of the other of TGT or TGC not selected for “c”, a codon for aspartic acid symbolically designated “d” consisting of one of GAT or GAC, a codon for aspartic acid symbolically designated “d*” consisting of the other of GAT or GAC not selected for “d”, a codon for glutamic acid designated “e” consisting of GAA or GAG, a codon for phenylalanine designated “f” consisting of one of TTT or TTC, a codon for phenylalanine designated “f” consisting of the other of TTT or TTC not selected for “f”, a codon for glycine designated “g” consisting of one of GGT or GGC, a codon for glycine designated “g” consisting of the other of GGT or GGC not selected for “g”; a codon for alanine designated “a” consisting of one of GCT or GCC. A codon for alanine designated “a*” consisting of the other of GCT or GCC not selected for “a”, and, arbitrarily but consistently and predictably selection of any other codon designated “b”.

### BRIEF DESCRIPTION OF THE DRAWINGS

FIG. 1 is a diagram showing the assembly of a signature containing vector.

FIG. 2 is a genetic map of the vector constructed according to the Example.

FIG. 3 is a hypothetical agarose gel showing the predicted position of various digestion fragments of the plasmid pGEM-MAZ-poem3 generated by restriction enzymes.

FIGS. 4A and 4B are gel tracings verifying insertion of the nucleic acid signature (Seq. No. 1) sequence into the vector depicted in FIG. 2.

### DETAILED DESCRIPTION OF THE PREFERRED EMBODIMENT

For more than fifty years, it has been known that the genetics for all living species is rooted in nucleic acids as expressed by the genetic code. In nature the code is universally based on combinations of four bases in DNA and four bases in RNA. These base sequences are arranged in triplets known as codons. All the amino acids which make up proteins are assigned to one or more codons. Since there are four bases arranged in triplets, mere are a total of 64 unique combinations of triplets. In biology, the rule of triplets must be obeyed. However, if a nucleic acid sequence is utilized for a non-genetic purpose, the rule of triplets, while providing a convenient reference point, does not necessarily apply. If triplets do not apply, then the number of unique combinations can be amplified, so that a quartet of four bases yields 256 unique combinations, a sequence of five yields 1024 unique combinations, and so on. Combination from triplets up to nanoplets are practical in scope since the latter would provide sufficient character assignments to express an Oriental language, but doublets only provide 16 character combinations. Thus, the length of individual codons may be increased from three nucleotides up to nine per codon, and the lengths of individual codons may be an even or uneven number. Additionally, codons may be positioned as an interrupted sequence or may be separated one from an other by defined noncoding separators.

The present invention is directed to the use of DNA or RNA sequences to impart or communicate social information. Social information encompasses language of any origin, music, notational systems, and can be expanded to the realm of mathematics and digital processing, so long as the total number of unique combinations is large enough to encode all the individual characters contained in the repertoire. For practical considerations and convenience a preferred embodiment of the invention focuses on triplets, to take advantage of the fact that most of the triplet codons are already assigned to amino acids, which by convention, are abbreviated to a single letter.

Table 1 shows the identity in nature of codons for each amino acid denoted by its single letter.

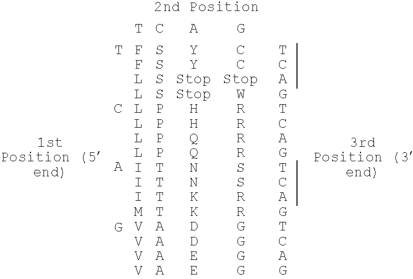

This is the starting point for assignment of letters to triplets. The degeneracy of the genetic code is evident in the multiple codons for many amino acids. In building the number of characters required for expression of English words and elements of punctuation, only up to two codons are selected for any letter. Those amino acids with multiple codons in excess of two (i.e. arginine with six) are then available for assignment to characters not represented in the natural scheme (i.e. b, j, o, x, and z).

Table 2 is an artificial coding table incorporating not more than two codons for amino acids of conventionally assigned letter (which is adopted in this embodiment), and assigning the remaining five letters and a number of punctuation elements to the rest of the codons.

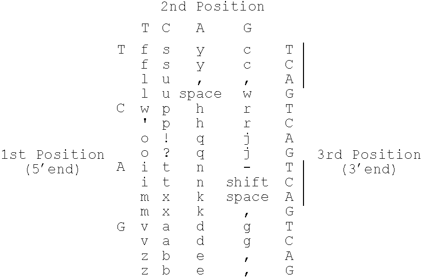

For example, referring to the second row of Table 2, two of the codons normally coding for arginine have been reassigned to “j”. The reason for adopting up to two codons for each letter, is that synthesizing a signature sequence may inadvertently introduce sites, such as an unwanted restriction endonuclease substrate site that would interfere with cloning and other genetic manipulations. There exist computer programs that scan sequences for such sites, and an appropriate codon substitution can be made. In a preferred embodiment, one or more uncommitted codons can function as a signal to a directory containing alternative assignments in the sequence immediately following it. In this way the inventory of characters can be expanded to include numbers and other symbols. This is especially important where, in music, a directory is required to specify tonality, clefts, harmonic and voice interactions and complementarity, amplitude, rhythm, and multiple instrumentation.

The signature sequence of the present invention is intended as a personal identifier, and may be a message of any desired length. In the situation in which the information-containing sequence is cloned into a host organism for preservation, there may be size limitations to the size of the insert. Such limitations are well known to those skilled in the genetics art. In addition to a signature identifier, which is readable using a computer program, the present invention may optionally include a second encrypted authenticating tag sequence, known only to the entity preparing the construct, in utilizing the 18 letters of the English alphabet which already have letter assignments, it is possible for the reader to reconstruct the message without resort to a coiling key. This is because whole messages can be constructed from the 18 characters having a universally recognized letter designation. For example, “START HERE”. This means that the reader who lays out the corresponding amino acid sequence derived from an open reading frame of DNA, will immediately be apprised that a message is contained in the sequence. Thus, it an object of the present invention not to hide messages, but make them more easily accessible and recognizable. In addition to these identifier and authenticating sequences, the present invention may also incorporate various amounts of DNA from any species into a cloning vehicle. For large amounts of DNA, particularly up to the size of the human genome, cloning may involve creating vast libraries. The techniques for creating such libraries are well known in the art. In one embodiment of the present invention there will be a signature identifier sequence, an optional authenticating rag sequence, and all or a portion of genomic DNA of the individual so identified. In addition, the construct may contain an indicator gene, which when expressed in a suitable host, will display a characteristic color or other detectable indicator trait. One example is the lux operon which causes host organisms to chemiluminesence when provided the proper substrate.

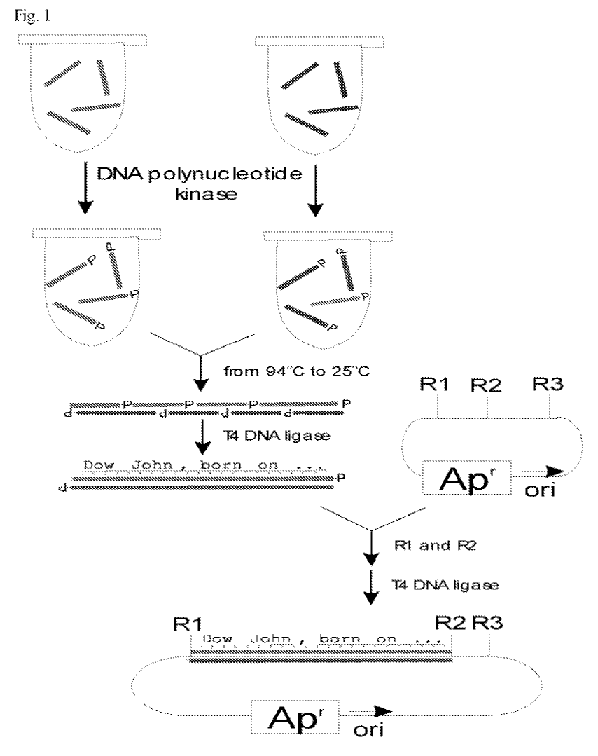

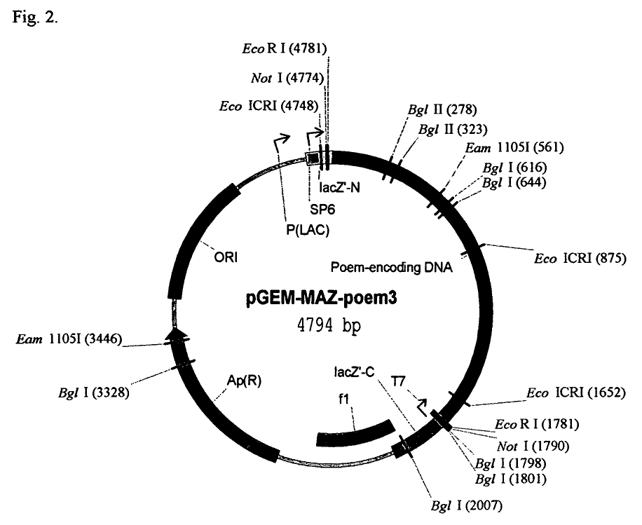

The general scheme for synthesizing a signature encoding nucleic acid sequence is shown in FIG. 1. The target sequence is divided into fragments suitable for chemical synthesis. The complementary sequence has also been divided into fragments of similar size. The primary strand is depicted on the left side of the diagram, and the complementary strand is depicted on the right. The location of the fragments is chosen so that the boundaries of two adjacent fragments from one strand lie approximately in the center of the fragment from the complementary fragment. The resulting set of oligonucleotides is depicted in the FIG. 2. All oligonucleotides in these sets are created using standard techniques known in the art. Note that portions of the oligonucleotides at the beginning and end of the sequence encode endonuclease restriction sites, to facilitate subsequent cloning info a host.

Further in the general scheme, the oligonucleotides are phosphorylated by treatment with Bacteriphage T4 polynucleotide kinase in the presence of ATP. The polynucleotide fragment sets are then mixed together in stoichiometric concentration at 94 degrees C. and gradually cooled to 25 degrees to permit annealing of the complementary strands. The nicks in the duplex DNA are then repaired with Bacteriophage T4 ligase. All procedures are carried out using standard, well-documented methods.

In FIG. 1, R1, R2, and R3 represent unique sites cleavable by the appropriate restriction endonuclease. The purpose of such cloning cassettes is not only to achieve insertion of the desired sequence, bid also, upon extraction of DNA from the host organism, to retreat the construct, and confirm that the size of PCR amplified fragments migrating on a agarose gel conform to the predicted size of the insert. The plasmid depicted in FIG. 1 is shown minimally to have an origin of replication (Ori), and a gene conferring ampicillin resistance as a selective marker (Ap^{r}) for isolation of successful transformants. Other selective markers include resistance to other antibiotics, nutritional restrictions, and suppressor tRNA genes. Advantages of the present invention will be ascertained by the Example that follows.

### EXAMPLE

The current example demonstrates how the nucleic acid can be converted into a time capsule for delivery social information through time, space and generations. A putative and somewhat fanciful signature encoding nucleic acid sequence, nevertheless representative, was synthesized from the following poem, utilizing the symbol assignments of Table 2:

A mooing cow the beginning,
The middle a trilling bird,
If pronounced correctly,
That is how my name is heard,
After my father's grandmother,
My grandmother-great,
Who had died just before I was born,
Right on that very same date,
Masha is just a shortening,
Maria is my name.

### Secondly Alexeyevna.

To tell from whom I came.

### Daughter of Alexey,

That's how in Russia it's done,
And with my eleven-letter last name,
I have very much fun.
Others cannot pronounce it,
But my tongue roles right along,
My full name put together,
Sounds to me like a song,

### Zdanovskaia

The nucleic acid sequence encoding this poem as constructed using artificial coding table presented in Table 2 is:

Sequence I.D. No. 1:
aaggtagagcgcctagatgctgctgatcaacggctagtgtctgtggtaga
cccacgagtaggcggagggcatcaacaacattaacggctgatagagcacC
cacgagtagatgatcgacgacttagagtaggcctagacccgcatcttatt
aatcaacggctaggcgatccgcgactgatagagcatcttctagccccgcc
tgaacctgtcgaactgcgaggactagtgcctgcgccgcgagtgcacctta
tactgatagagcacccacgccacctagatctcctagcacctgtggtagat
gtactagaacgccatggagtagatctcctagcacgaggcccgcgactgat
agagcgccttcaccgagcgctagatgtactagttcgccacccacgagcgc
ctctcctagggccgcgccaacgacatgctgacccacgagcgctgatagag
catgtactagggccgcgccaacgacatgctgacccacgagcgcagtggcc
gcgaggccacctgatagagctggcacctgtagcacgccgactaggacatc
gaggactagcggccgtccacctaggcggagttcctgcgcgagtagagcat
ctagtgggcctcctaggcgctgcgcaactgatagagccgcatcggccaca
cctagctgaactagactcatgctacttaggttgagcgttattagtccgct
atggaataggatgctactgaatgatagagcatggcttctcatgcttagat
ttcttagcgatcatctacttaggcctagtctcatctacgtactgaaaata
ttaatggttgatagagcatggctcgtattgcttagatttcttagatgtat
tagaatgctatggaatgatagagctctgaatgtctaaatgatttgtatta
gagcgctttggaaacagaatacgaagttaatgcttgatagagcactctat
agactgaattgttgtagtttcgtctaatgtagtggcatctaatgtagagc
atttagtgtgccatggaatgatagagcgatgcttcaggtcatactgaacg
ttagctattttagagcgctttggaaacagaatattgatagagcactcatg
ctactctctcttagcatctatggtagattaattagagccgttcatcttct
attgcttagattactctctcttaggatctaaatgaatgatagagcgctaa
tgattagtggattactcattagatgtattaggaattggaagttgaaaata
gtttggaaaccaccgagcgctagttggcctccacctagaacgccatggag
tgatagagcatctagcacgccgtcgagtaggtcgagcgctactagatgtc
gtgccactagttctcgaactgatagagcctaacccacgagcgctcctagt
gcgccaacaacctgacctagccccgcctgaacctgtcgaactgtgagtag
atcacctgatagagcgcgtcaacctagatgtactagaccctgaacggctc
ggagtagcgcctgttggagtcctagcgcatcggccacacctaggccttgc
tgaacggctgatagagcatgtactagttctcgttgttgtagaacgccatg
gagtagccctcgacctagaccctgggcgagacccacgagcgctgatagag
ctccctgtcgaacgactcctagaccctgtagatggagtagttgatcaagg
agtaggcctagtccctgaacggctaatagagcgtggacgccaacctggtc
tccaaggccatcgcctaataggat

The artificial coding table of the current example was build on the basis of the biological coding table. Therefore, anyone who will apply the biological coding table to translation of the appropriate coding frame will receive the corresponding letter sequence:

Sequence I.D. No: 2

*SA*MLLING*CLW*THE*AEGINNING**STHE*MIDDLE*A*TRILLI
NG*AIRD**SIF*PRLNLSNCED*CLRRECTLY**STHAT*IS*HLW*MY
*NAME*IS*HEARD**SAFTER*MY*FATHERLS*GRANDMLTHER**SM
Y*GRANSMLTHERSGREAT**SWHL*HAD*DIED*RSST*AEFLRE*SE*
WAS*ALRN**SRIGHT*LN*THAT*VERY*SAME*DATE**SMASHA*IS
*RSST*A*SHLRTENING**SMARIA*IS*MY*NAME**SSECLNDLY*S
ALETEYEVNA**STL*TELL*FRLM*WHLM*SI*CAME**SDASGHTER*
LF*SALETEY**STHATLS*HLW*IN*SRSSSIA*ITLS*DLNE**SAND
*WITH*MY*ELEVENSLETTER*LAST*NAME**SI*HAVE*VERY*MSC
H*FSN**SLTHERS*CANNLT*PRLNLSNCE*IT**SAST*MY*TLNGSE
*RLLES*RIGHT*ALLNG**SMY*FSLL*NAME*PST*TLGETHER**SS
LSNDS*TL*ME*LIKE*A*SLNG**SVDANLVSKAIA*

A brief analysis of this sequence will reveal the unusual for the native sequence feature—the presence of elements of human language (highlighted in green), thus making the reviewer aware of the artificial nature of the sequence and the presence of social information encoded by the sequence. To demonstrate the lack of elements of human language in the frames not carrying social information the same sequence is translated in two additional frames:

Sequence I.D. No. 3

KVERLDAADQRLVSVVDPRVGGGHQQH*RLIEHPRVDDRRLRVGLDPHLI
NQRLGDPRLIEHLLAPPEPVELRGLVPAPRVHLILIEHPRHLDLLAPVVD
VLERHGVDLLAPGPRLIERLHRALDVLVRHPRAPLLGPRQPHADPRALIE
HVLGPRQRHADPRAQWPRGHLIELAPVARRLGHRGLAVVHLGGVPAPVEH
LVGLLGAAQLIEPHRPHLAELDSCYLG*ALLVRYGIGCY*MIEHGFSCLD
FLAIIYLGLVSSTY*KY*WLIEHGSYCLDFLDVLECYGMIEL*MSK*FVL
EPFGNRIPS*CLIEHSID*IVVVSSNVVASNVEHLVCHGMIEPCFRSY*T
LAILERFGNRILIEHSCYSLLASMVD*LEPFIFYCLDYSLLGSK*MIER*
*LVDYSLDVLGIGS*K*FGNHRALVGLHLERHGVIEHLARRRVGRALLDV
VPLVLELIEPNPRALLVRQQPDLAPPEPVEL*VDHLIERVNLDVLDPERL
GVAPVGVLAHRPHLGLAERLIEHVLVLVVVERHGVALDLDPGRDPRALIE
LPVERLLDPVDGVVDQGVGLVPERLIERGRQPGLQGHRLIG

Sequence I.D. No. 4

GPAPRCC*STASVCGRPTRRRASTTLTADRAPTSR*STT*SRPRPASY*S
TARRSATDRASSSPA*TCRTARTSACAASAPYTDRAPTPPRSPSTCGRCT
RTPQSRSPSTRPATDRAPSPSARCTSSPPTSASPRAAPTTC*PTSADRAC
TRAAPTTC*PTSAVAARPPDRAGTCSTPTRTSPTSGRPPRRSSCASRASS
GPPRRCATDRAASATPS*TRLMLLRLSVISPLWNPMLLNDRAWLLMLRFL
SDHLLPPSLIYVLKILMVDPAWLVLLRFLRCIRMLWNDRALNV*MICIRA
LWKQNTKLMLDRALYRLNCCSFV*CSGI*CRAFSVPWNDPAMLQVILNVS
YFRALWKQNIDRALMLLSLSIYGRLIRAVHLLLLRLLSLRI*MNDRALMI
SGLLIRCIRNWKLKIVWKPPSASWPPPRTPWSDRASSTPSSRSSATRCRA
TSSRTDRA*PTSAPSAPTT*PSPA*TCRTVSRSPDRARQPRCTRP*TAPS
SACWSPSASATPRPC*TADPACTSSRCCRTPWSSPRPRPWARPTSADPAP
CRTTPRPCRWSS*SRSRPSP*TANRAWTPTWSPRPSPNR

Once becoming aware of special features of the analyzed nucleic acid, with little effort the reviewer will be able to reconstruct the artificial coding table as well as the entire social information encoded into the nucleic acid.

This signature nucleic acid sequence was cloned into a vector plasmid following the general scheme set forth in FIG. 1. The resulting plasmid was designated pGEM-MAZ-poem 3. This plasmid is depicted by its map in FIG. 2. Note that the plasmid contains an origin of replication, and multiple cloning sites, including two EcoRI restriction sites strategically placed to con iris insertion of the signature sequence, and a selective marker for ampicillin resistance (Ap^{r}). The highlighted portion of the map shows the position of the insert. Further details of the process include the conventional procedures of phosphorylation of oligonucleotides with T4 polynucleotide kinase at 37 degrees C. for one hour in 50 mM TrisHCl buffer, pH 7.5, 10 mM MgCl₂, 5 mM DTT, and 1 mM ATP. Then all oligonucleotides were added in stoichiometric quantities to the same reaction mixture where they were first heated to 94 and then were slowly cooled down to room temperature. Nicks were repaired with Bacteriophage T4 ligase in the presence of 2 mM ATP for three hours. Analysis of the composition of the resulting mixture performed by means of gel electrophoresis reveal the presence of DNA sequences of varying size. The isolation of the required sequence was performed by means of polymerase chain reaction using primers Z1 and A36;

Sequence I.D. No 5:
aaggtagagcgcctagatgccgctgatcaacggctagtgtctgtggcaga
Sequence I.D. No 6:
Atcctattaggcgatggccttggagaccaggttggcgtccacgctctatt

Upon completion of the reaction the reaction mixture was subjected to the agarose gel electrophoresis and the DNA fragment of the required size ( ) was purified from the gel and was cloned into commercially available plasmid vector pGEM-T Easy Vector. Many other plasmids, cosmids, and vectors may be used to clone a signature and other sequences into respective permissive hosts. For general enabling references to the techniques and methods available in the art to carry out the genetic manipulations involved in die present invention, consult “Current Protocols in Molecular Biology”, vol. 1, ed. F. Ausubel, et al. (John Wiley & Sons, Inc.: 1987-1994), and Maniatis, “A Laboratory Manual of Molecular Biology” (J. T. Baker: 1982).

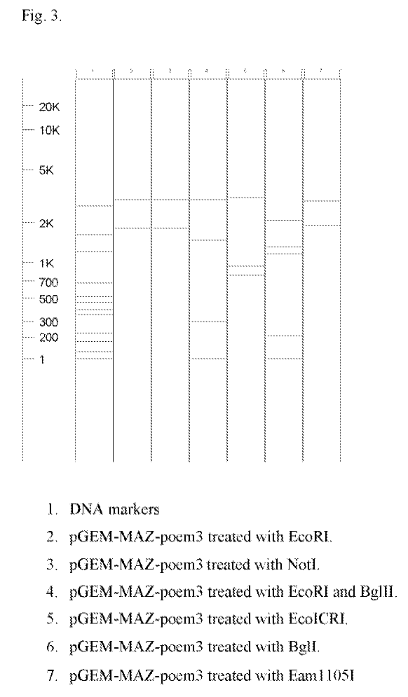

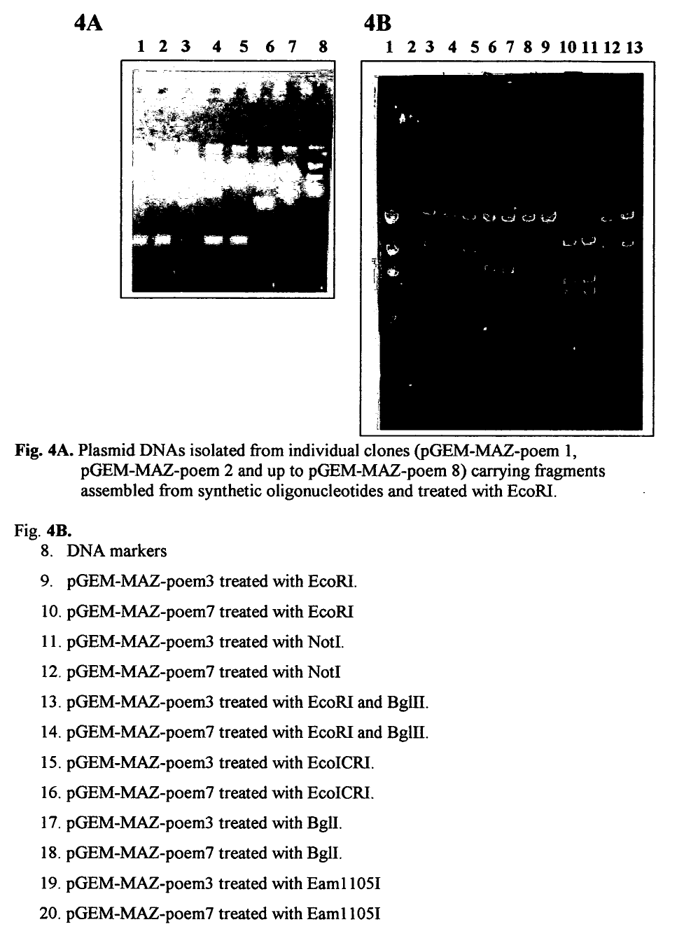

Subsequent to cloning of the signature sequence, the poem, host cells were grown up and the plasmid DNA was extracted and digested with various restriction endonucleases known to have substrate sequences present in the plasmid DNA, FIG. 3 shows a hypothetical tracing of where on an agarose gel the appropriate bands of amplified polynucleotides are expected to migrate, if successful integration of the signature sequence is achieved. The actual gels, shown in FIGS. 4 and 5, confirm that polynucleotide fragments of the expected size are displayed, especially EcoRI fragments flanking the insert are seen in FIG. 4A, Please note the two bands of slightly differing, but expected, molecular weight in lane 3 of the gel. Also note the position of bands 3 and 7 in FIG. 4B for the relative size of the polynucleotides obtained by digestion with two other restriction endonucleases.

What is claimed is:

1. A signature encoding nucleic acid language sequence containing a message formed from Setters and elements of punctuation of codon identities of the genetic code comprising
a continuous sequence of nucleotide triplets, wherein each triplet in an open reading frame corresponds to at least one and not more than two degenerate codons for a triplet encoded amino acid having single letter symbol assigned by convention to such letter, and all other letters and all punctuation elements are assigned randomly from the remaining amino acid regenerate codons, and non-amino acid encoding codons, in an internally consistent usage; and
spliceable sequences 5′ and 3′ of said signature encoding nucleic acid nucleic acid language sequence enabling integration into a vector for propagation and genetic preservation.

2. The signature encoding nucleic acid language sequence of claim 1, wherein said sequence contains an encrypted tag identifier sequence.

3. The nucleic acid sequence of claim 1 insertable into the vector of claim 1 comprising
a signature encoding nucleic acid language sequence
cloning cassettes positioned 3′ and 5′ of said nucleic insertable sequence containing at least one site recognizable by a restriction endonuclease; and
a fragment of chromosomal DNA of any species sought to be genetically preserved, positioned 3′ of said signature encoding nucleic acid sequence.

4. A signature encoding nucleic acid language base of characters comprising a sequence constructed by
selecting codons having conventionally designated single letter symbols assigned to encoded amino acids including
a codon for phenylalanine, symbolically designated “f” consisting of one or not more than two of TIT and TTC, a codon for amino acid leucine, symbolically designated “l”, consisting of one or not more than two of TTA, TTG, CTT, CTC, CTA, and CTG, a codon for isoleucine, symbolically designated “l”, consisting of one and not more than two of ATT, ATC, and ATA, a codon for methionine, symbolically designated “m”, consisting uniquely of ATG, a codon for valine, symbolically designated “v”, consisting of one or not more than two of GTT, GTC, GTA, and GTG, a codon for serine, symbolically designated “s”, consisting of one or not more than two of TCT, TCC, TCA, AGT, and AGC, a codon for proline, symbolically designated “p”, consisting of one or not more than two of CCT, CCC, CCA, and CCG, a codon for threonine, symbolically designated “f”, consisting of one or note more than two of ACT, ACC, AC A, and ACG, a codon for alanine, symbolically designated “a”, consisting of one or not more than two of GCT, GCC, GCA, and GCG, a codon for tyrosine, symbolically designated “y”, consisting of one or not more than two of TAT and TAC, a codon for histidine, symbolically designated “h”, consisting of one or not more than two of CAT and CAC, a codon for glutamine, symbolically designated “q”, consisting of one or not more than two of CAA and CAG, a codon for asparagine, symbolically designated “n”, consisting of one or not more than two of AAT and A AC, a codon for glutamic acid, symbolically designated “e”, consisting of one or not more than two of GAA and GAG, a codon for cysteine, symbolically designated “c”, consisting of one or not more than two of TGT and TGG, a codon for tryptophane, symbolically designated “w”, consisting uniquely of TGG, a codon for arginine, symbolically designated “r”, consisting of one or not more than two of COT, CGC, CGA, CGG, AGA, and AGG, a codon for glycine, symbolically designated “g”, consisting of one or not more than two of GGT, GGC, GGA, and GGG; and
a codon selected arbitrarily and consistently from all remaining codons and assigned to a character consisting of “b”, “j”, “o”, “x” and “z” or to an element of punctuation, “space”, “!”, “,”, “.”, “upper case”, “″”, “?” and “-”.

5. A signature encoding nucleic acid music base comprising a sequence constructed by selecting codons having conventionally designated single letter symbols assigned to encoded amino acids corresponding to the notes of a music scale including
A codon for cystine symbolically designated “c” consisting of one of TGT or TGC, a codon for cystine symbolically designated “c*” consisting of the other of TGT or TGC, a codon for aspartic acid symbolically designated “d” consisting of one of GAT or GAC, a codon for aspartic acid symbolically designated “d*” consisting of the other of GAT or GAC, a codon for glutamic acid designated “e” consisting of GAA or GAG, a codon for phenylalanine designated “f” consisting of one of TTT or TTC, a codon for phenylalanine designated “f” consisting of the other of TTT or TTC, a codon for glycine consisting of one of GGT or GGC, a codon for glycine designated “g” consisting of the other of GGT or GGC, a codon for alanine designated “a” consisting of one of GCT or GCC, a codon for alanine designated “a” consisting of the other of GCT or GCC, and the codon for methionine designated consisting uniquely of ATG.


SEQUENCE LISTING (101 sequences)

SEQ ID NO: 1 - DNA, 1774 bp, Artificial
  synthetic sequence
aaggtagagcgcctagatgctgctgatcaacggctagtgtctgtggtagacccacgagtaggcggagggcatcaacaacattaacggctgatagagcacccacgagtagatgatcgacgacttagagtaggcctagacccgcatcttattaatcaacggctaggcgatccgcgactgatagagcatcttctagccccgcctgaacctgtcgaactgcgaggactagtgcctgcgccgcgagtgcaccttatactgatagagcacccacgccacctagatctcctagcacctgtggtagatgtactagaacgccatggagtagatctcctagcacgaggcccgcgactgatagagcgccttcaccgagcgctagatgtactagttcgccacccacgagcgcctctcctagggccgcgccaacgacatgctgacccacgagcgctgatagagcatgtactagggccgcgccaacgacatgctgacccacgagcgcagtggccgcgaggccacctgatagagctggcacctgtagcacgccgactaggacatcgaggactagcggtcgtccacctaggcggagttcctgcgcgagtagagcatctagtgggcctcctaggcgctgcgcaactgatagagccgcatcggccacacctagctgaactagactcatgctacttaggttgagcgttattagtccgctatggaataggatgctactgaatgatagagcatggcttctcatgcttagatttcttagcgatcatctacttaggcctagtctcatctacgtactgaaaatattaatggttgatagagcatggctcgtattgcttagatttcttagatgtattagaatgctatggaatgatagagctctgaatgtctaaatgatttgtattagagcgctttggaaacagaatacgaagttaatgcttgatagagcactctatagactgaattgttgtagtttcgtctaatgtagtggcatctaatgtagagcatttagtgtgccatggaatgatagagcgatgcttcaggtcatactgaacgttagctattttagagcgctttggaaacagaatattgatagagcactcatgctactctctcttagcatctatggtagattaattagagccgttcatcttctattgcttagattactctctcttaggatctaaatgaatgatagagcgctaatgattagtggattactcattagatgtattaggaattggaagttgaaaatagtttggaaaccaccgagcgctagttggcctccacctagaacgccatggagtgatagagcatctagcacgccgtcgagtaggtcgagcgctactagatgtcgtgccactagttctcgaactgatagagcctaacccacgagcgctcctagtgcgccaacaacctgacctagccccgcctgaacctgtcgaactgtgagtagatcacctgatagagcgcgtcaacctagatgtactagaccctgaacggctcggagtagcgcctgttggagtcctagcgcatcggccacacctaggccttgctgaacggctgatagagcatgtactagttctcgttgttgtagaacgccatggagtagccctcgacctagaccctgggcgagacccacgagcgctgatagagctccctgtcgaacgactcctagaccctgtagatggagtagttgatcaaggagtaggcctagtccctgaacggctaatagagcgtggacgccaacctggtctccaaggccatcgcctaataggat

SEQ ID NO: 2 - protein, 6 aa, Artificial
  synthetic sequence
MLLING

SEQ ID NO: 3 - protein, 9 aa, Artificial
  synthetic sequence
AEGINNING

SEQ ID NO: 4 - protein, 4 aa, Artificial
  synthetic sequence
STHE

SEQ ID NO: 5 - protein, 6 aa, Artificial
  synthetic sequence
MIDDLE

SEQ ID NO: 6 - protein, 8 aa, Artificial
  synthetic sequence
TRILLING

SEQ ID NO: 7 - protein, 4 aa, Artificial
  synthetic sequence
AIRD

SEQ ID NO: 8 - protein, 10 aa, Artificial
  synthetic sequence
PRLNLSNCED

SEQ ID NO: 9 - protein, 9 aa, Artificial
  synthetic sequence
CLRRECTLY

SEQ ID NO: 10 - protein, 5 aa, Artificial
  synthetic sequence
STHAT

SEQ ID NO: 11 - protein, 4 aa, Artificial
  synthetic sequence
NAME

SEQ ID NO: 12 - protein, 5 aa, Artificial
  synthetic sequence
HEARD

SEQ ID NO: 13 - protein, 6 aa, Artificial
  synthetic sequence
SAFTER

SEQ ID NO: 14 - protein, 8 aa, Artificial
  synthetic sequence
FATHERLS

SEQ ID NO: 15 - protein, 11 aa, Artificial
  synthetic sequence
GRANDMLTHER

SEQ ID NO: 16 - protein, 17 aa, Artificial
  synthetic sequence
GRANDMLTHERSGREAT

SEQ ID NO: 17 - protein, 4 aa, Artificial
  synthetic sequence
SWHL

SEQ ID NO: 18 - protein, 4 aa, Artificial
  synthetic sequence
DIED

SEQ ID NO: 19 - protein, 4 aa, Artificial
  synthetic sequence
RSST

SEQ ID NO: 20 - protein, 6 aa, Artificial
  synthetic sequence
AEFLRE

SEQ ID NO: 21 - protein, 4 aa, Artificial
  synthetic sequence
ALRN

SEQ ID NO: 22 - protein, 6 aa, Artificial
  synthetic sequence
SRIGHT

SEQ ID NO: 23 - protein, 4 aa, Artificial
  synthetic sequence
THAT

SEQ ID NO: 24 - protein, 4 aa, Artificial
  synthetic sequence
VERY

SEQ ID NO: 25 - protein, 4 aa, Artificial
  synthetic sequence
SAME

SEQ ID NO: 26 - protein, 4 aa, Artificial
  synthetic sequence
DATE

SEQ ID NO: 27 - protein, 6 aa, Artificial
  synthetic sequence
SMASHA

SEQ ID NO: 28 - protein, 4 aa, Artificial
  synthetic sequence
RSST

SEQ ID NO: 29 - protein, 10 aa, Artificial
  synthetic sequence
SHLRTENING

SEQ ID NO: 30 - protein, 6 aa, Artificial
  synthetic sequence
SMARIA

SEQ ID NO: 31 - protein, 4 aa, Artificial
  synthetic sequence
NAME

SEQ ID NO: 32 - protein, 9 aa, Artificial
  synthetic sequence
SSECLNDLY

SEQ ID NO: 33 - protein, 11 aa, Artificial
  synthetic sequence
SALETEYEVNA

SEQ ID NO: 34 - protein, 4 aa, Artificial
  synthetic sequence
TELL

SEQ ID NO: 35 - protein, 4 aa, Artificial
  synthetic sequence
FRLM

SEQ ID NO: 36 - protein, 4 aa, Artificial
  synthetic sequence
WHLM

SEQ ID NO: 37 - protein, 4 aa, Artificial
  synthetic sequence
CAME

SEQ ID NO: 38 - protein, 9 aa, Artificial
  synthetic sequence
SDASGHTER

SEQ ID NO: 39 - protein, 7 aa, Artificial
  synthetic sequence
SALETEY

SEQ ID NO: 40 - protein, 7 aa, Artificial
  synthetic sequence
STHATLS

SEQ ID NO: 41 - protein, 7 aa, Artificial
  synthetic sequence
SRSSSIA

SEQ ID NO: 42 - protein, 4 aa, Artificial
  synthetic sequence
ITLS

SEQ ID NO: 43 - protein, 4 aa, Artificial
  synthetic sequence
DLNE

SEQ ID NO: 44 - protein, 4 aa, Artificial
  synthetic sequence
SAND

SEQ ID NO: 45 - protein, 4 aa, Artificial
  synthetic sequence
WITH

SEQ ID NO: 46 - protein, 13 aa, Artificial
  synthetic sequence
ELEVENSLETTER

SEQ ID NO: 47 - protein, 4 aa, Artificial
  synthetic sequence
LAST

SEQ ID NO: 48 - protein, 4 aa, Artificial
  synthetic sequence
NAME

SEQ ID NO: 49 - protein, 4 aa, Artificial
  synthetic sequence
HAVE

SEQ ID NO: 50 - protein, 4 aa, Artificial
  synthetic sequence
VERY

SEQ ID NO: 51 - protein, 4 aa, Artificial
  synthetic sequence
MSCH

SEQ ID NO: 52 - protein, 7 aa, Artificial
  synthetic sequence
SLTHERS

SEQ ID NO: 53 - protein, 6 aa, Artificial
  synthetic sequence
CANNLT

SEQ ID NO: 54 - protein, 9 aa, Artificial
  synthetic sequence
PRLNLSNCE

SEQ ID NO: 55 - protein, 4 aa, Artificial
  synthetic sequence
SAST

SEQ ID NO: 56 - protein, 6 aa, Artificial
  synthetic sequence
TLNGSE

SEQ ID NO: 57 - protein, 5 aa, Artificial
  synthetic sequence
RLLES

SEQ ID NO: 58 - protein, 5 aa, Artificial
  synthetic sequence
RIGHT

SEQ ID NO: 59 - protein, 5 aa, Artificial
  synthetic sequence
ALLNG

SEQ ID NO: 60 - protein, 4 aa, Artificial
  synthetic sequence
FSLL

SEQ ID NO: 61 - protein, 4 aa, Artificial
  synthetic sequence
NAME

SEQ ID NO: 62 - protein, 8 aa, Artificial
  synthetic sequence
TLGETHER

SEQ ID NO: 63 - protein, 7 aa, Artificial
  synthetic sequence
SSLSNDS

SEQ ID NO: 64 - protein, 4 aa, Artificial
  synthetic sequence
LIKE

SEQ ID NO: 65 - protein, 4 aa, Artificial
  synthetic sequence
SLNG

SEQ ID NO: 66 - protein, 12 aa, Artificial
  synthetic sequence
SVDANLVSKAIA

SEQ ID NO: 67 - protein, 27 aa, Artificial
  synthetic sequence
KVERLDAADQRLVSVVDPRVGGGHQQH

SEQ ID NO: 68 - protein, 199 aa, Artificial
  synthetic sequence
RLIEHPRVDDRRLRVGLDPHLINQRLGDPRLIEHLLAPPEPVELRGLVPAPRVHLILIEHPRHLDLLAPVVDVLERHGVDLLARGPRLIERLHRALDVLVRHPRAPLLGPRQRHADPRALIEHVLGPRQRHADPRAQWPRGHLIELAPVARRLGHRGLAVVHLGGVPARVEHLVGLLGAAQLIEPHRPHLAELDSCYLG

SEQ ID NO: 69 - protein, 11 aa, Artificial
  synthetic sequence
ALLVRYGIGCY

SEQ ID NO: 70 - protein, 24 aa, Artificial
  synthetic sequence
MIEHGFSCLDFLAIIYLGLVSSTY

SEQ ID NO: 71 - protein, 24 aa, Artificial
  synthetic sequence
WLIEHGSYCLDFLDVLECYGMIEL

SEQ ID NO: 72 - protein, 12 aa, Artificial
  synthetic sequence
FVLERFGNRIRS

SEQ ID NO: 73 - protein, 8 aa, Artificial
  synthetic sequence
CLIEHSID

SEQ ID NO: 74 - protein, 29 aa, Artificial
  synthetic sequence
IVVVSSNVVASNVEHLVCHGMIERCFRSY

SEQ ID NO: 75 - protein, 27 aa, Artificial
  synthetic sequence
TLAILERFGNRILIEHSCYSLLASMVD

SEQ ID NO: 76 - protein, 17 aa, Artificial
  synthetic sequence
LEPFIFYCLDYSLLGSK

SEQ ID NO: 77 - protein, 4 aa, Artificial
  synthetic sequence
MIER

SEQ ID NO: 78 - protein, 13 aa, Artificial
  synthetic sequence
LVDYSLDVLGIGS

SEQ ID NO: 79 - protein, 64 aa, Artificial
  synthetic sequence
FGNHRALVGLHLERHGVIEHLARRRVGRALLDVVPLVLELIEPNPRALLVRQQPDLAPPEPVEL

SEQ ID NO: 80 - protein, 109 aa, Artificial
  synthetic sequence
VDHLIERVNLDVLDPERLGVAPVGVLAHRPHLGLAERLIEHVLVLVVVERHGVALDLDPGRDPRALIELPVERLLDPVDGVVDQGVGLVPERLIERGRQPGLQGHRLIG

SEQ ID NO: 81 - protein, 7 aa, Artificial
  synthetic sequence
GRAPRCC

SEQ ID NO: 82 - protein, 27 aa, Artificial
  synthetic sequence
STASVCGRPTRRRASTTLTADRAPTSR

SEQ ID NO: 83 - protein, 8 aa, Artificial
  synthetic sequence
SRPRPASY

SEQ ID NO: 84 - protein, 16 aa, Artificial
  synthetic sequence
STARRSATDRASSSPA

SEQ ID NO: 85 - protein, 75 aa, Artificial
  synthetic sequence
TCRTARTSACAASAPYTDRAPTPPRSPSTCGRCTRTPWSRSPSTRPATDRAPSPSARCTSSPPTSASPRAAPTTC

SEQ ID NO: 86 - protein, 16 aa, Artificial
  synthetic sequence
PTSADRACTRAAPTTC

SEQ ID NO: 87 - protein, 58 aa, Artificial
  synthetic sequence
PTSAVAARPPDRAGTCSTPTRTSRTSGRPPRRSSCASRASSGPPRRCATDRAASATPS

SEQ ID NO: 88 - protein, 75 aa, Artificial
  synthetic sequence
TRLMLLRLSVISPLWNRMLLNDRAWLLMLRFLSDHLLRPSLIYVLKILMVDRAWLVLLRFLRCIRMLWNDRALNV

SEQ ID NO: 89 - protein, 29 aa, Artificial
  synthetic sequence
MICIRALWKQNTKLMLDRALYRLNCCSFV

SEQ ID NO: 90 - protein, 4 aa, Artificial
  synthetic sequence
CSGI

SEQ ID NO: 91 - protein, 62 aa, Artificial
  synthetic sequence
CRAFSVPWNDRAMLQVILNVSYFRALWKQNIDRALMLLSLSIYGRLIRAVHLLLLRLLSLRI

SEQ ID NO: 92 - protein, 66 aa, Artificial
  synthetic sequence
MNDRALMISGLLIRCIRNWKLKIVWKPPSASWPPPRTPWSDRASSTPSSRSSATRCRATSSRTDRA

SEQ ID NO: 93 - protein, 10 aa, Artificial
  synthetic sequence
PTSAPSAPTT

SEQ ID NO: 94 - protein, 4 aa, Artificial
  synthetic sequence
PSPA

SEQ ID NO: 95 - protein, 20 aa, Artificial
  synthetic sequence
TCRTVSRSPDRARQPRCTRP

SEQ ID NO: 96 - protein, 19 aa, Artificial
  synthetic sequence
TARSSACWSPSASATPRPC

SEQ ID NO: 97 - protein, 46 aa, Artificial
  synthetic sequence
TADRACTSSRCCRTPWSSPRPRPWARPTSADRAPCRTTPRPCRWSS

SEQ ID NO: 98 - protein, 7 aa, Artificial
  synthetic sequence
SRSRPSP

SEQ ID NO: 99 - protein, 18 aa, Artificial
  synthetic sequence
TANRAWTPTWSPRPSPNR

SEQ ID NO: 100 - DNA, 50 bp, Artificial
  synthetic sequence
aaggtagagcgcctagatgctgctgatcaacggctagtgtctgtggtaga

SEQ ID NO: 101 - DNA, 50 bp, Artificial
  synthetic sequence
atcctattaggcgatggccttggagaccaggttggcgtccacgctctatt